(19) United States
(12) Patent Application Publication
(10) Pub. No.: US 2017/0055496 A1
(43) Pub. Date: Mar. 2, 2017

(54) ANIMAL HEALTH SENSOR SYSTEM

(71) Applicant: Horse Sense Shoes, LLC, Plymouth, MN (US)

(72) Inventors: Michael McHugh, Plymouth, MN (US); Roger Roisen, Minnetrista, MN (US)

(21) Appl. No.: 15/351,159

(22) Filed: Nov. 14, 2016

Related U.S. Application Data
(63) Continuation of application No. PCT/US2015/030609, filed on May 13, 2015.
(60) Provisional application No. 61/992,498, filed on May 13, 2014.

(51) Int. Cl. A01K 11/00; A61B 5/00; A61B 5/024; A61B 5/11; A01K 29/00; A61B 5/01

(52) U.S. Cl. CPC A01K 11/008; A01K 29/005; A61B 5/01; A61B 5/024; A61B 5/11; A61B 5/6833

(57) ABSTRACT

A system includes a temperature sensor and circuitry to collect information from the temperature sensor and transmit the information to a server. The temperature sensor and circuitry are formed as an integrated node adapted to be placed on an exposed skin area of a tail, or on a hoof wall of an animal. The server processes the information and may transmit the information to one or more remote devices. A strain gage may also be included in the node to attach to the hoof wall.

### RELATED APPLICATION

This application is a continuation under 35 U.S.C. 111(a) from Internatinal Application No. PCT/US2015/030609, filed May 13, 2015, which claims priority to U.S. Provisional Application Ser. No. 61/992,498 (entitled Animal Health Sensor System, filed May 13, 2014) which applications are incorporated herein by reference.

### BACKGROUND

Prior systems attempted to measure physiological properties of various animals, but typically involved sensors coupled to remote transponders. This resulted in separate placement of the sensors and transponders, increasing the complexity of using such devices. The information collected from such sensors was stored for later analysis.

### SUMMARY

A system includes a physiological property sensor, and circuitry to collect information from the physiological property sensor and transmit the information to a server, wherein the physiological property sensor and circuitry are formed as an integrated patch adapted to be placed to sense a physiological property on a skin area on a tail of an animal.

A system includes a physiological property sensor, and circuitry to collect information from the physiological property sensor and transmit the information to a server, wherein the physiological property sensor and circuitry are formed as an integrated patch adapted to be placed to sense a physiological property on a hoof wall of an animal.

A system includes a server, a temperature sensor to sense ambient temperature about an animal and provide data representative of the sensed ambient temperature to the server, a physiological property sensor, and circuitry to collect information from the physiological property sensor and transmit the information to the server, wherein the physiological property sensor and circuitry are formed as an integrated node adapted to be placed on a tail of the animal to sense temperature of the animal, and wherein the server is a particular machine programmed to compile received information, adjust the sensed animal temperature as a function of sensed ambient temperature, and send communications to a device with a display.

A method includes receiving at a server, sensed temperature information via a sensor coupled to a skin area on a tail of an animal, receiving ambient temperature information corresponding to an ambient temperature about the animal, and processing the received sensed temperature information by the server to adjust the sensed temperature as a function of an ambient temperature about the animal.

### BRIEF DESCRIPTION OF THE DRAWINGS

FIG. 1A is a schematic block diagram of a veterinarian wrapped patch a according to an example embodiment.

FIG. 1B is a schematic block diagram of an adhesive attached patch according to an example embodiment.

FIG. 1C is a schematic block diagram of a strap attached patch according to an example embodiment.

FIG. 1D is a block diagram representation of different positions or postures of a horse according to an example embodiment.

FIG. 1E is a user interface graph of sensed information according to an example embodiment.

FIG. 2 is a block schematic diagram view of a wireless multiple sensing system according to an example embodiment.

FIG. 3 is a block diagram of a wireless sensor according to an example embodiment.

FIG. 4 is a block diagram of a wireless sensor according to an example embodiment.

FIG. 5A is a block diagram illustrating a track environment incorporating wireless sensors according to an example embodiment.

FIG. 5B is a block diagram illustrating a system incorporating wireless sensors according to an example embodiment.

FIG. 6 is a block diagram of a patch wireless sensor system according to an example embodiment.

FIG. 7 is a block diagram illustrating a disposable multiple sensor wireless sensor patch according to an example embodiment.

FIG. 8 is a block side view of the disposable multiple sensor wireless sensor patch according to an example embodiment.

FIG. 9 is a block diagram of a stable type environment incorporating wireless sensors according to an example embodiment.

FIG. 10A is a block diagram illustrating a tracking interface according to an example embodiment.

FIG. 10B is a chart illustrating posture information according to an example embodiment.

FIG. 11 is a block diagram illustrating a multiple diverse sensor system according to an example embodiment.

FIG. 12 is a web page based interface for selecting animals being monitored and providing posture information according to an example embodiment.

FIG. 13 is a web page based interface for selecting animals being monitored and providing additional information according to an example embodiment.

FIG. 14 is a web page based interface for selecting animals being monitored and providing hoof temperature and posture information according to an example embodiment.

FIG. 15 is a web page based interface providing tail sensor based posture information such as lying down or standing up according to an example embodiment.

FIG. 16 is a mobile web page based interface for selecting animals being monitored according to an example embodiment.

FIG. 17 is a mobile web page based interface illustrating temperature and position information for a selected animal according to an example embodiment.

FIG. 18 is an example computer system for implementing one or more methods or algorithms according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software or a combination of software and human implemented procedures in one embodiment. The software may consist of computer executable instructions stored on computer readable media such as memory or other type of storage devices. Further, such functions correspond to modules, which are software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system.

Various embodiments include one or more of sensors for pressure and temperature affixed on a tail, hoof, or on the body of a horse or other animal to be monitored. Further animals include for example, cattle, sheep, goats, and other livestock. Sensed physiological properties or parameters are transmitted via a wireless RF radio. The data is collected and uploaded to a server. The server data may be charted over time and available via the website. In one embodiment, a strain gauge is connected to a resistor and a supply voltage such as VCC that may be provided by a battery. Resistor divider output is monitored with a data convertor—analog to digital convertor and a sampled voltage is converted to a binary number with a microprocessor. The microprocessor converts the number to a digital signal that is then moved to the RF radio for transmission. The transmitted data may be further transmitted to a server via a network. The server processes the data and provide for display of the data on a website viewable by a client device, such as a computer, including mobile devices.

The sensor and circuitry may be integrated in one embodiment to form a thin circuit board. The circuit board may be supported on a patch suitable for fixing to the tail or on the hoof wall. In one embodiment, the patch is an adhesive patch that may be flexible and placed on the underside of the tail where there is a hairless skin area. Such a position provides a more consistent measurement than an area with hair, as direct contact with the skin places the sensor or sensors directly against the skin without any intervening medium. Temperature signatures are likely much more accurate and consistent with such a placement. In one embodiment the patch may be adhered to the tail to place the sensor in contact with the skin area on an underside of the tail.

In various embodiments, the patch has an adhesive suitable for providing retentive contact with the skin or hoof wall. The patch may also be wrapped around the hoof wall or tail, or strapped into place around the hoof wall or tail. Many owners tail wrap the horses' tails before competitive events, and the patch may be held in place by such a wrap. Also, Broodmares tails are wrapped when they are nearing labor, providing a suitable mechanism for coupling the patch to the skin areas of the tail.

The outside of the hoof wall placement of the patch is useful for measuring an increase in heat in this area associated with an early sign of laminitis. It is important to catch laminitis in the early stages. Heat in the hoof wall occurs 12 hours or more before any signs that the horse would visibly show in its posture.

In one embodiment, the temperature measured by the temperature sensor in the patch coupled to the tail may be compared with both ambient temperature and rectal temperature to obtain an accurate correlation of rectal temperature to the sensor temperature. By taking contemporaneous measurements of the tail, ambient, and rectal temperature, an algorithm may be created, or a table lookup mechanism may be created to provide such a correlation. For instance, the tail temperature may run lower than the rectal temperature when ambient temperatures are lower than the sensor data, and may run higher when the ambient temperature is higher. A table of ambient temperatures may provide corresponding corrections to the measured temperature to derive the rectal temperature.

Ambient temperature may be measured about the horse, such as in a stall, or a field where the horse is located. The location of a temperature sensor to measure ambient temperature should be similar the location when first determining the algorithm or lookup table as well as when used later to use the algorithm or lookup table to adjust the temperature reading from the patch. In one embodiment, the ambient temperature sensor should be positioned to best correlate to the ambient temperature the animal is subjected to.

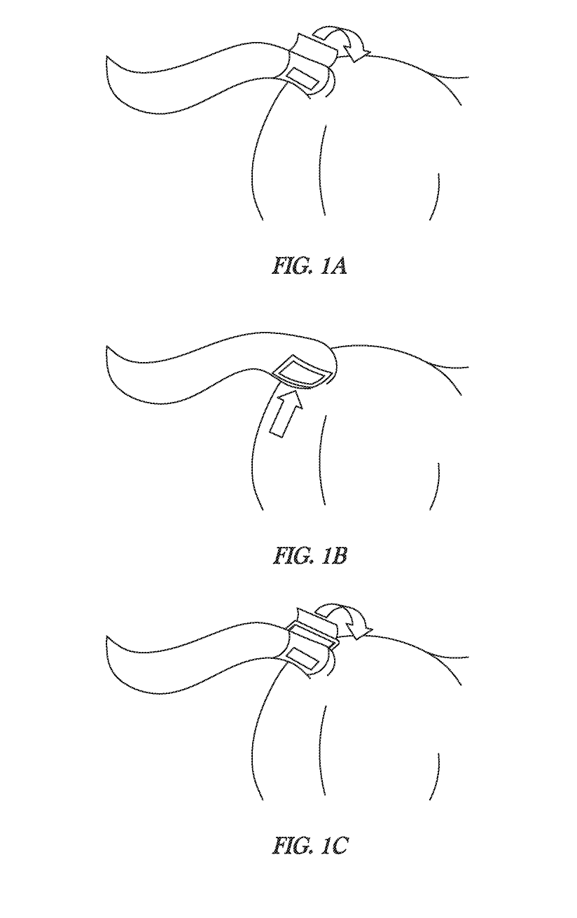

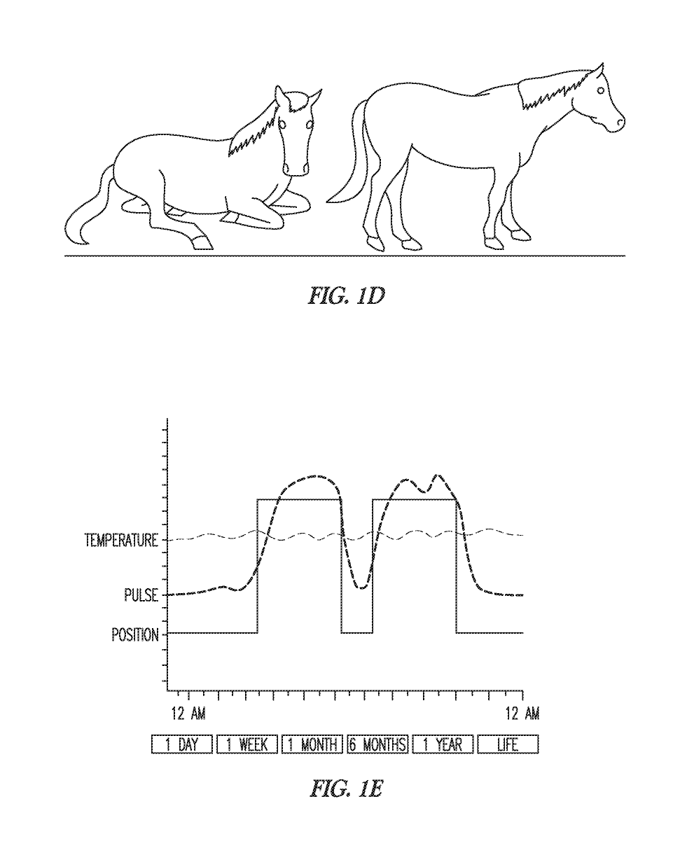

The following FIGS. 1A, 1B, and 1C illustrate different attachment mechanisms described above corresponding to a vet wrapped attachment option, an adhesive attachment option, and a strapped attachment option respectively. Buckles, tape, ties, clips, hook and loop, and other fastening mechanisms may be used with or without an adhesive backed wrap in various embodiments.

The integrated sensor and circuitry may be formed within an enclosure or pad to protect them. Nodes may transmit to a server independently for example, via a ZigBee IEEE 802 network or other type of network device providing a suitable communication range in a compact format. In some embodiments, multiple nodes may be networked with animal collecting and buffering data from the other nodes and transmitting it to the server. The sensor and circuitry of a node may be encapsulated in plastic or other material and sealed, such as hermetically sealed to protect it from harsh environmental conditions.

In one embodiment, the patch may include an accelerometer. The accelerometer may provide data indicative of postural changes, such as when an animal changes position from standing to laying down as shown in FIG. 1D. The accelerometer data may indicate a change in vertical as well as a rotating or turning motion of the animal corresponding to the animal lowering itself to the ground and then rolling on its side. The rolling motion may also be detected by a gyroscope. Such accelerometer readings may produce different patterns that are easily identifiable as a laying down movements. Standing back up from a lying position may also produce the opposite pattern. Lowering to a kneeling or lying on belly position may similarly be detected via accelerometer measurements.

A pulse sensor may also be included in further embodiments. When all are incorporated in a patch fastened to exposed skin under the tail of a horse, data may be collected to provide temperature, pulse, and position of the horse as illustrated in an example screen display of a graph in FIG. 1E. Hovering with a cursor or finger in the case of a touch screen display over each individual plot line will cause the scale for that line to be displayed. The position line for example is fairly intuitive. In the lower portions of the line, the horse is lying down. In the upper portions of the line, the horse is standing. Transitions between the lower and upper portions illustrate the timing of lying down or standing up. Touching the pulse line causes the vertical axis of the graph to display numbers for the pulse rate. Touching the temperature line causes the vertical axis to display temperature values. The x-axis illustrates time, which as shown corresponds to a time period between 12 AM and 12 PM. The time period may be adjusted in further embodiments to show measurements over longer and shorter periods of time, such as minutes, hours, days, weeks, months, years, or life time. Some corresponding view buttons are illustrated and may be selected to show the data for a corresponding period. Pattern matching may be performed in further embodiments to correlate known events, such as gestation, sickness, etc., to current data to provide predictive information regarding the health or status of the horse.

In various embodiments, each node includes at least one sensor and circuitry, such as a microprocessor and transceiver or radio. There may be one or more such sensors and circuitry on each hoof wall and/or tail. Data is collected by the microprocessor and uploaded via a wireless network to a server. The data may be buffered and sent in bursts in some embodiments. The server may be coupled to the Internet in various embodiments. Each independent set of sensors may upload data, or a lead sensor and circuitry may collect all data from the horse or other animal and upload the collected information. In further embodiments, applications may include sensors gathering other data for example a diagnostic patch on the horse may be reading and collecting data of heart beat rate or breathing rate, or oxygen saturation and uploading to the internet. Strain gauges may be used on various parts of the hoof in further embodiments.

In some embodiments, the server provides a real time website with a graphical user interface to allow a user to view data for one or more horses and to quickly display data for each individual hoof. Historical data for a horse may also be processed and compared to current data. Trend analysis may be performed to generate alerts if the trends indicate undesired changes in pressure or other measured property. Alerts may also be generated when a particular sensor reading such as temperature meets a selected threshold. Pattern matching may be performed in further embodiments to determine if current patterns in temperature or postural behavior when compared to previous patterns. Such undesired patterns may also result in alerts. The alerts may take the form of an SMS message or other form of instant communication such as Twitter™ messages.

Advantages of various embodiments may include one or more of knowing when a horse is sick, and keeping track of multiple horses. Knowing when a horse is in heat and when the horse is ready to give birth. The health history of the horse may also be observed. Knowing when the horse races best can also be of value, as well as having early signs of potential injury providing the ability to stop further injury. The recovery rate of a horse may also be observed. All of this information can be of benefit in performing preventive medical care and in obtaining an managing insurance on horses.

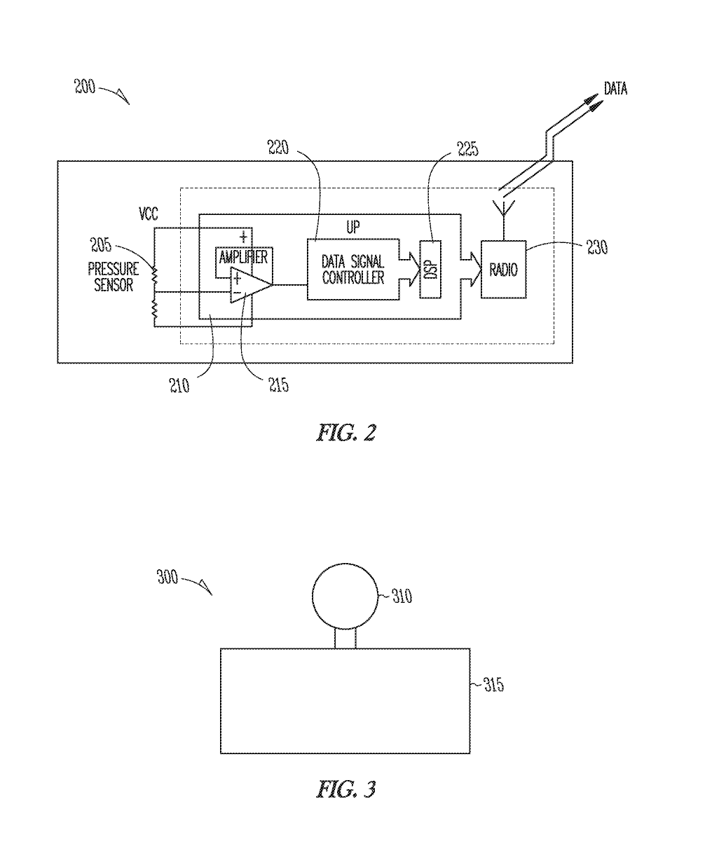

FIG. 2 is a block schematic diagram view of a wireless sensor node 200 according to an example embodiment. One or more sensors 205 may be one or many different types of sensing devices such as temperature sensor, a strain gauge, or strain sensitive resistor 205 in a resistive divider coupled to circuitry 210. Circuitry 210 includes an amplifier 215 coupled to a microprocessor 220 and digital signal processor 225. Sampled signals from the sensor 205 are processed to provide a signal representative of the sensed strain. The signal is transmitted by a radio 230 along with an ID that may be used to uniquely identify the animal, and the precise location of the node on the animal. In further embodiments, a unique node ID may be transmitted, and later correlated with information to identify the location of the node on the animal. In one embodiment, the node 200 is in the form of a patch containing wireless sensors 205 according to an example embodiment. In one embodiment, the sensors may include one or more of a gyroscope, accelerometer, temperature sensor, and magnetometer.

The node 200 comprising integrated sensor 205 and circuitry 210 may be formed within an enclosure or pad to protect them as illustrated in FIG. 3 at 300. FIG. 3 is a block diagram of a wireless sensor node according to an example embodiment. In one embodiment, the node includes a sensing element 310 and circuitry 315. The sensor may be placed on the tail as described above. The sensor and circuitry of a node may be encapsulated in plastic or other material and sealed, such as hermetically sealed to protect it from harsh environmental conditions.

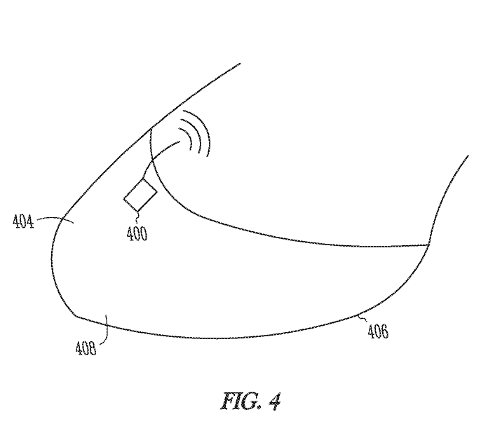

FIG. 4 is a block diagram of a wireless sensor 400 according to an example embodiment. In one embodiment, the sensor 400 is coupled to a wall 404 of a hoof 406. The sensor 400 may be a temperature sensor or a strain gauge used to sense stress or cracks occurring in the hoof, such as quarter cracks in the quarter 408 of hoof 406. In further embodiments, both types of sensors may be included. The wireless sensor 400 transmits signals representative of the strain or temperature or both.

The strain gauge 415 may provide an overall measure of strain in the horseshoe as the horse or other animal moves. In one embodiment, the strain is measured along the length of a horseshoe to hoof interface. The strain gauge 415 may include circuitry to process and transmit signals representative of the strain. In further embodiments, one or more nodes may contain global positioning system components to provide location information. Location information may also be provided based on one or more triangulation techniques in further embodiments.

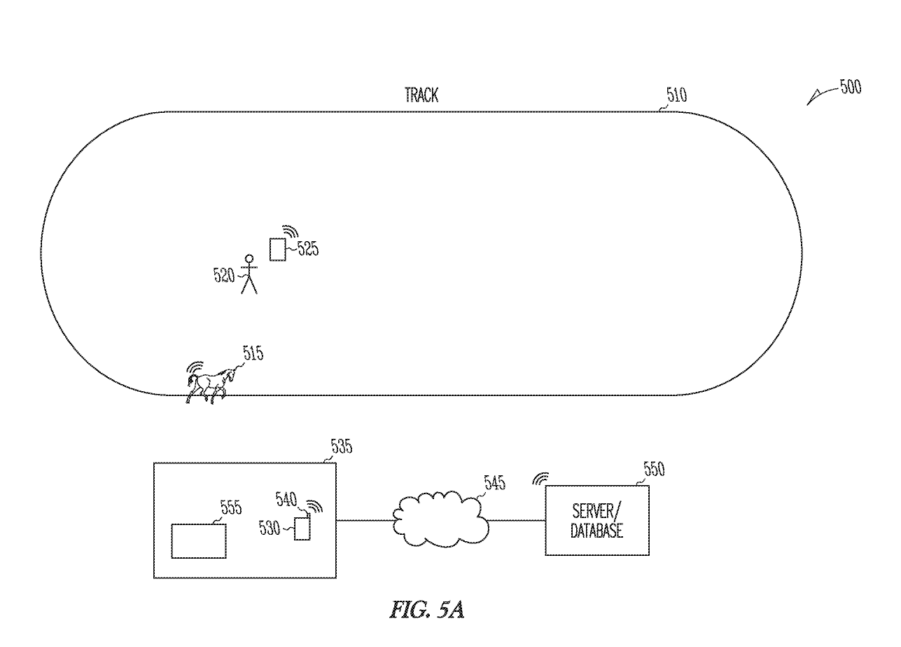

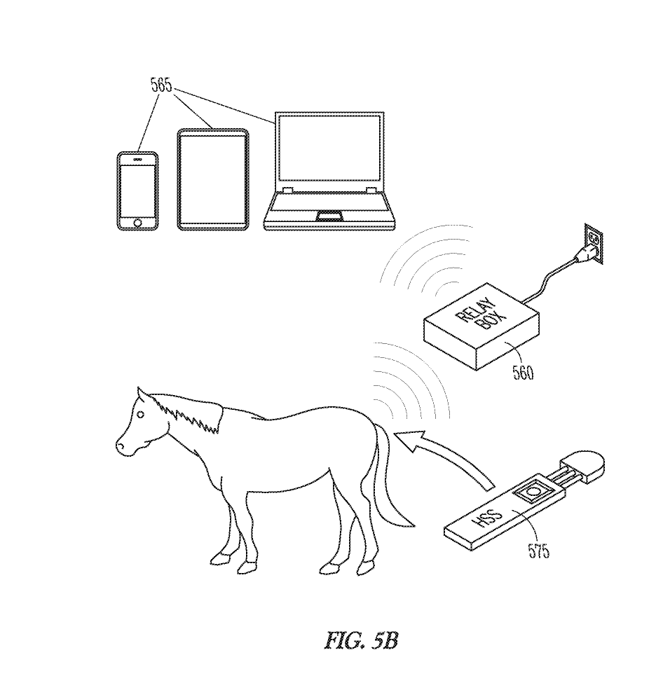

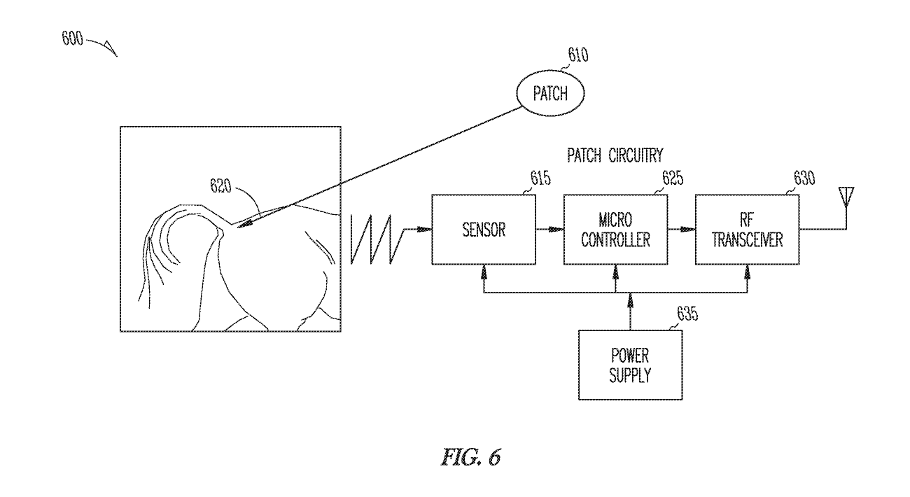

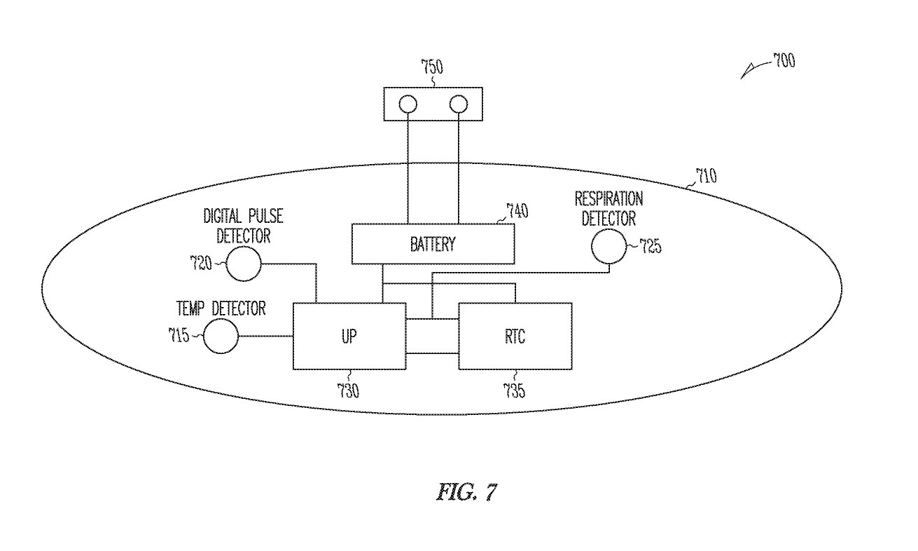

FIG. 5A is a block diagram illustrating a track environment 500 incorporating wireless sensors according to an example embodiment. A training track 510 is shown with a horse 515 that is equipped with one or more sensors, including pressure, temperature, strain, and other sensors that transmit signals representative of physiological properties of a horse or other animal. A trainer is indicated at 520, along with a mobile wireless device 525 that contains information from the sensors.

In one embodiment, the sensor information is transmitted to one sensor on the animal 515, and then is received by a collection device or system 530 in a building, such as a barn or stable 535. The system 530 need not be located in the barn in various embodiments, and may be comprised of a computer system such as a laptop or other type of computer that receives transmissions from the sensors. Thus it should be located within range of animal being monitored. When there are instances that the animal is out of range the information may be saved on the microprocessor memory and sent to the muter, seconds, minutes, hours or days later after the animal is back within range of the collection device. In one embodiment, a universal serial bus (USB) type of receiver 540 or a wireless router may be employed to receive the signals transmitted from the sensor nodes, or primary node on the animal. The system 530 is coupled to a network 545, such as the Internet, to provide the sensed data to a server 550. The server 550 may include a database, and programming to compile the data for each horse.

An ambient temperature sensor 555 is shown to obtain ambient temperature measurements corresponding to the environment the horse is being subjected to. The temperature measurements may be used to calibrate temperature measurements taking by sensors coupled to the horse, such as via a tail based temperature sensor in a patch or wrap coupled to the exposed skin under the tail of the horse. In further embodiments, the ambient temperature sensor may be located in or near other areas where the horse may be located, such as stall, pasture, training, and hospital environments. In still further embodiments, ambient temperature may be obtained from web based services, using locations closest or most representative of the location of the horse. Adjustments to the tail sensed temperature may then be made using such an ambient temperature reading obtained and may be further adjusted by which environment the horse is currently in.

The data may be sent from the server 550 through one or more wireless networks to the mobile device 525 to provide information about the horse being monitored. The wireless device may either access a secure account on the server corresponding to the horses being trained by the trainer 520, or in some embodiments, location information of the mobile device and of the horses or system 530 may be provided to the server 550 and used to access information likely to be relevant to the trainer 520 or horse handler, vet Ferrier, or other person or persons.

In some embodiments, data is collected as the animal runs on the track 510, or in any environment such as for example paddock, jump course, or any other recreational, sporting, riding, or grazing environment. The data may include pressure, shock, temperature, strain, and other information. Shock information may be obtained if the sampling frequency is high enough to measure expected ranges of shock. In further embodiments, a node may include an accelerometer to provide the shock data. The data may be time stamped, provided with an ID to identify either the node, or the animal and location on the animal, optionally buffered, optionally processed, and transmitted to the system 530. As transmission ranges of the nodes increases, the nodes may themselves couple directly to a network and send the data directly to server 550. The information may then be processed and compiled to provide real time stride information for the animal being monitored.

In some embodiments, the direction information may be used to inform a jockey to switch leads down the stretch of a run around the track. Such information can be utilized in a range of training and competitive situations to enhance performance techniques unique to a particular animal such as a horse. Collected information can be utilized with video of animal activities to better recognize performance indicators.

In one embodiment, the sampling is performed at a programmable rate, and may be varied in response to training needs and to conserve node battery life. Typical sampling rates may range up to several thousand samples per second. In some embodiments, the gait of the horse may be measured and used to reprogram the sampling for an accelerometer sensor in real time to coincide with hoof contact with the ground. Higher sampling rates may be utilized during contact, with lower rates being used when no contact is expected. Temperature sensing sampling may occur less frequently, as various temperatures are not expected to change rapidly. Temperatures may be sensed on the order of seconds per sample or minutes between samples in some embodiments.

In some embodiments, the collected data at each node corresponds to physiological parameter measurements at desired sampling rates. The collected data may be time stamped and sent by each node to a collection point. The collection point may be one of the nodes on an animal, or may be a transceiver located within range of the normal locations of the animal. In some embodiments, the collected data at each sample point is packaged and sent to a server for processing and display of individual sensor data or a combination of one or more sensor's data for one point in time. The data may also be buffered at a node or the collection point, and statistically processed to provide averages over time for each sensor's data.

In further embodiments, multiple physiological properties are transmitted from the same sensing device or node through a single transmission means, and displayed graphically to show contemporaneous relationships between the properties.

FIG. 5B is an alternative embodiment illustrating the use of a relay box 560 to receive data from sensors and transmit the data to one or more user devices indicated at 565. In one embodiment, the relay box 560 may plug into grid power, or may be battery powered. The relay box 560 receives signals from up to 256 sensors 575 coupled to one or more horses 570. The sensors may be coupled to the underside of a horse tail as previously described. The relay box 560 may include processing power to perform the analytics and also communicate with devices 565 via one or more different networks, including for example WiFi and cellular networks in different embodiments. The relay box 560 may also couple to a remote server for providing backup services or analytics and communications with the user devices 565.

Setting up the system in FIG. 5B may involve utilizing on the devices 565 to log into a server web site by entering a name, a horse's name, an associate tag serial number, and an SSID code from the sensor 575. The relay box 560 may then be powered up, and a power button on the sensor 575 pushed to power on the patch/sensor. The devices may then pair with the relay box and begin collecting data. The data may be selected periodically in some embodiments. A user may also request real time data between the periodic collections in some embodiments.

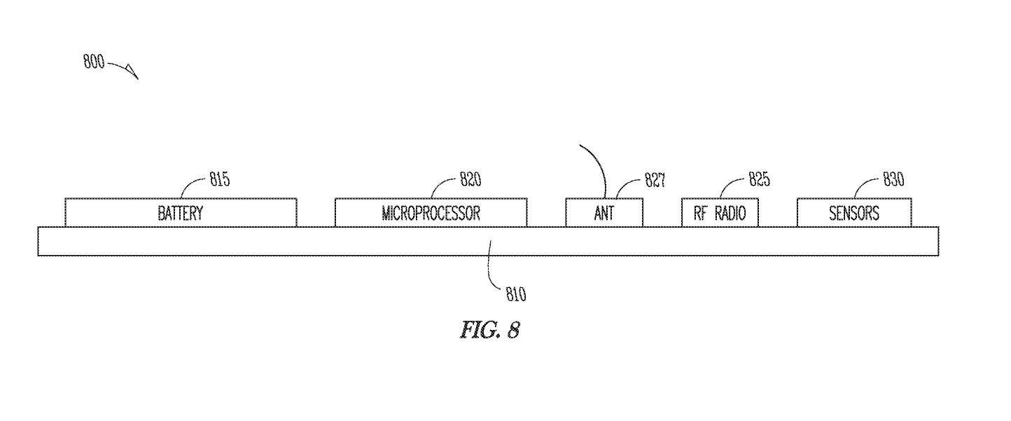

FIG. 6 is a block diagram of a patch wireless sensor system 600 according to an example embodiment. A patch 610 node having one or more sensors 615 is positioned on a desired portion of an animal as indicated at 620. The sensor provides data to a micro-controller 625 which processes the data and provides the data to an RF transceiver 630 for transmission. A power supply 635 is provided to provide power to the active components. In various embodiments, sensors 615 may include one or more of a magnetometer, gyroscope, altimeter, and one or more accelerometers. The magnetometer may be used to measure direction of the horse. The altimeter may be an atmospheric pressure sensor to determine the altitude of the horse. The altimeter may be sensitive enough to detect elevation changes corresponding a horse laying down or standing. The accelerometers may be oriented different to detect movements in different directions.

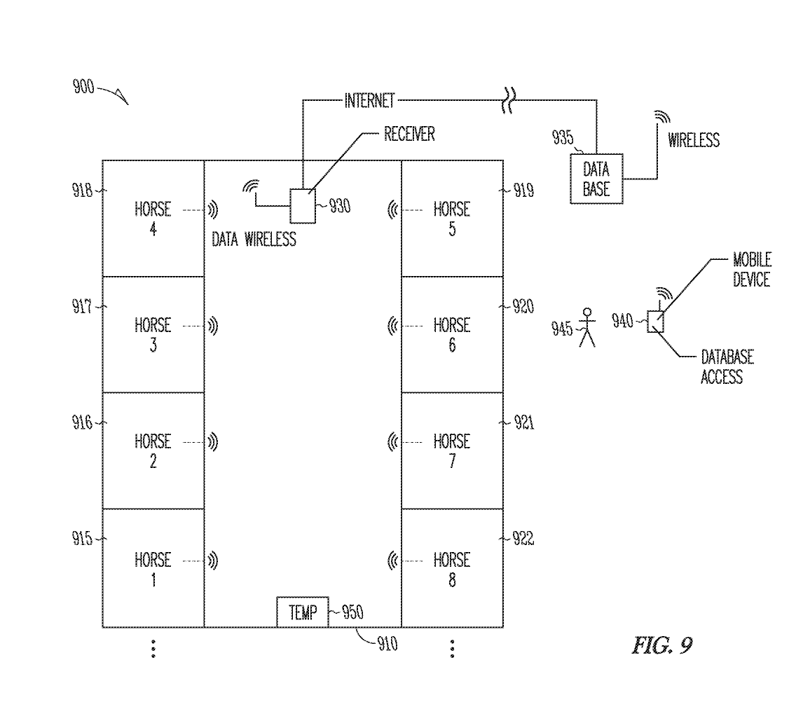

FIG. 7 is a block diagram illustrating a disposable multiple sensor wireless sensor patch 700 node according to an example embodiment. Patch 710 includes a temperature sensor 715 and a sensor 720 to detect one or more of direction, acceleration, pulse, oxygen, altitude, and movement in various embodiments. Typical sensors include one or more of pedometer, pulse detector, oxygen sensor, gyroscope, magnetometer, altimeter, and accelerometers. A respiration detector 725 may also be included and positioned proximate the chest of the horse to detect movements associated with breathing. Each provides signals to a microprocessor 730 via appropriate analog to digital conversion if necessary. A real time clock (RTC) 736 provides time information corresponding to the time of the measurements. A battery 740 provides power to the active components, including a transceiver that provides a unique radio address identifier as indicated at transceiver 750. In some embodiments, the transceiver 750 may be located on the patch 710, or near or adjacent the patch. The transceiver transmits information regarding the node and time stamped data collected. In further embodiments, a GPS or triangulation based location function may be added to the patch and position information may also be transmitted.

FIG. 8 is a block side view of a disposable multiple sensor wireless sensor patch 800 according to an example embodiment. The patch includes an adhesive substrate 810 supporting a battery 815, microprocessor 820, RF radio or transceiver 825, associated antenna for sending and receiving transmissions, and one or more sensors 830. In further embodiments, the patch 800 may be secured to the animal tail or hoof wall in several different ways, including but not limited to separate adhesives, straps, clips, sewing, or other methods.

FIG. 9 is a block diagram of a stable type environment 900 incorporating wireless sensors according to an example embodiment. In one embodiment, the stables are designed for horses as indicated by several stalls, each corresponding to a horse indicated at 915, 916, 917, 918, 919, 920, 921, and 922. Each of the horses may have one or more nodes for sensing various properties. A receiver 930 is positioned within the environment 910, or within range of the nodes to receive information transmitted from the nodes. In some embodiments, it is convenient to position the receiver 930 close to the nodes to reduce power requirements and conserve battery life. Some nodes may include transceivers that can receive power setting information to ensure they transmit at a power level that can be reliably received by the receiver 930.

The receiver 930 transmits the collected data to a database and server 935. Server 935 may provide web pages or other format information to one or more mobile devices 940 for viewing by a user or trainer 945. In further embodiments, the server 935 may provide the information to any type of network appliance for interaction with a user.

Typical properties that are monitored by the nodes include pressure, temperature, pulse, and respiration. In further embodiments, the server 935 may transmit signals to induce a stimuli. The stimuli may be provided by nodes that include devices to provide electric current, shock wave therapy, mechanical massage, medications via pumps or topically. Some nodes may include transceivers and may be dedicated to delivering one or more of the stimuli when activated via the server. The server may receive command from the trainer to provide the stimuli, or may be programmed to provide stimuli automatically if defined parameter thresholds are met or exceeded. An ambient temperature sensor 950 may also be located in the stable and provide ambient temperature data to the server 935 via receiver 930.

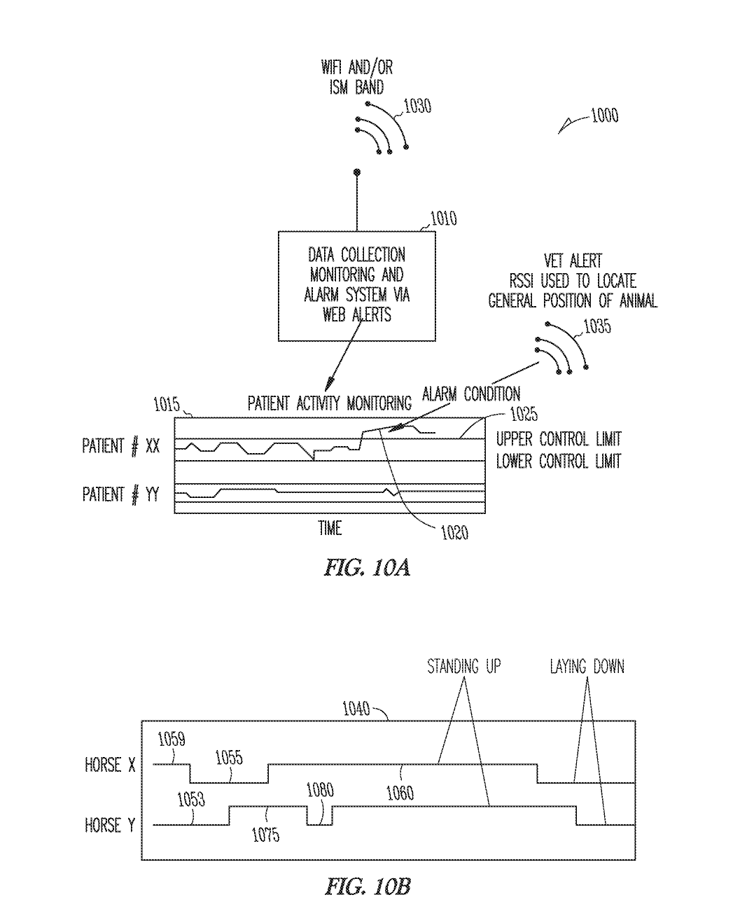

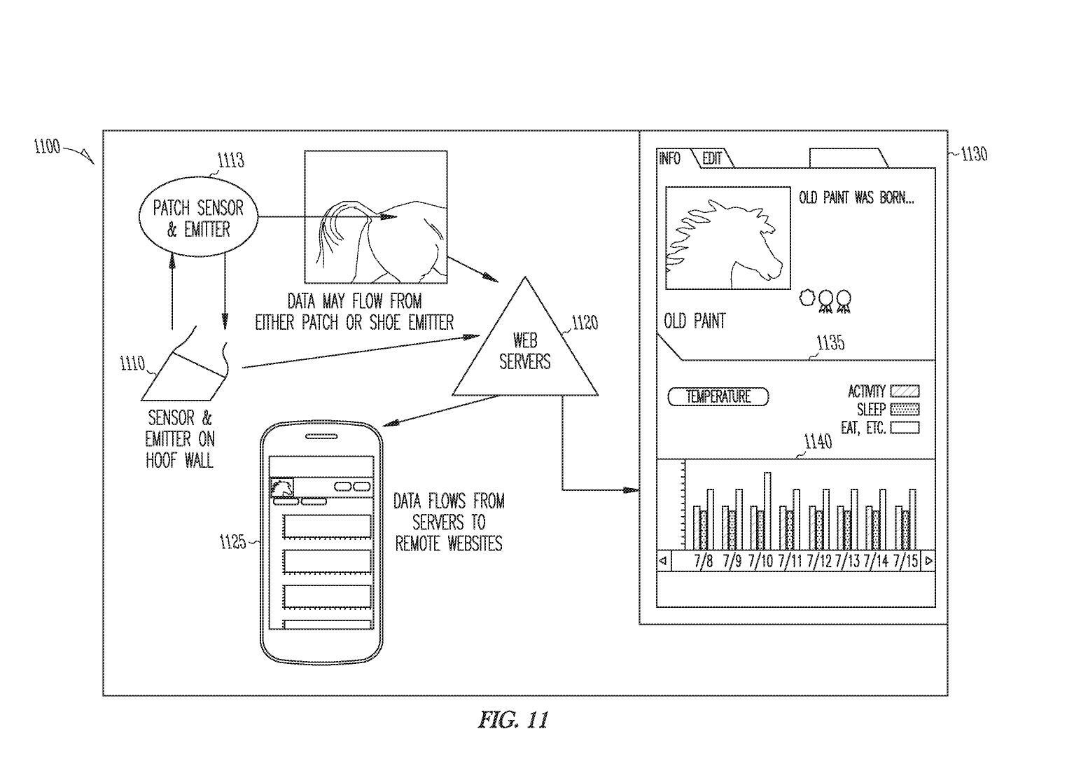

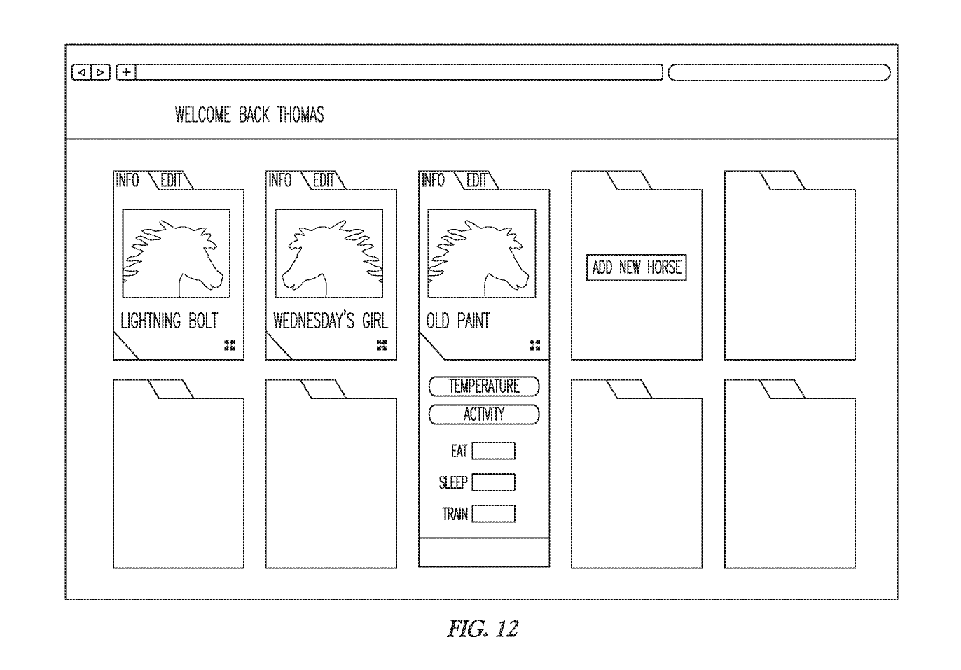

FIG. 10A is a block diagram illustrating a tracking interface 1000 according to an example embodiment. In one embodiment, alarms are provided via a data collection, monitoring, and alarm system 1010 via web alerts. Animals, referred to as patients in this example have a property being monitored as indicated at a chart 1015. The value of the property over time is indicated at 1020. When the property value exceeds an upper control limit 1025, an alert is sent as indicated at 1030. The alert may be received by a device being used by the trainer to alert the trainer or veterinarian. In one embodiment, received signal strength indicator (RSSI) 1035 may be used to provide a general location of the animal. In further embodiments, GPS or triangulation may be used. In further embodiments, accelerometer, gyroscope, orientation, or digital compass sensors may be included in nodes to identify stride analysis. The location of the animal may be crucial if the animal is sick, or may need medical attention. In some embodiments, the properties being monitored may be indicative of gestation and associated labor. Rather than keeping an animal confined, the monitoring may provide sufficient notice to retrieve the animal and provide appropriate medical care.

FIG. 10B is a chart 1040 illustrating the posture of two horses, horse x represented by line 1050 and horse y represented by line 1053. The number of horses viewable in chart 1040 may vary between one and as many as can be monitored and will fit on a display and be reasonably viewable. The display may be scrollable in further embodiments to accommodate tracking of more horses than fit on a display screen being used. Horse x corresponding to line 1050 can be seen as laying down at 1055 and standing at 1060. Horse y corresponding to line 1053 is seen standing at 1075 and laying down at 1080.

FIG. 1 is a block diagram illustrating a multiple diverse sensor system 1100 according to an example embodiment. System 1100 includes nodes on one or more patch nodes 1113, such as on a horse 1114 or hoof wall 1110. The data may from all or one selected node in various embodiments to a web server or servers 1120. The data is processed, and provided to one or more network appliances 1125, 1130 for viewing and interaction with a user such as a trainer. Information may be presented to a user of the appliance in the form of an activity indicator at 1135 indicating the activity of a selected horse. Old Paint, in this example. A chart 1140 may also be presented to illustrate activity.

FIG. 12 is a web page based interface for selecting animals being monitored and providing hoof temperature information according to an example embodiment. The web page in this example provides links to information related to three different horses being tracked, as well as the ability to add a new horse to be monitored. In the page shown, a horse named “Old Paint” has been selected, and a diagram illustrating the activity and temperature information is provided.

In further embodiments, in further embodiments, multiple physiological properties may be displayed to show contemporaneous relationships between the properties. For example, position changes, such as those detected by the accelerometer, on their own might not trigger concern, and neither would temperature changes on their own. But temperature and position (laying down vs standing up) changes together (at the same time) might trigger concern in a trainer. All, or selected portions of physiological data collected from the animal may be displayed together to provide an overall picture of the state of the animal at a particular time, or over a desired time frame. The data may be statistically processed to provide averages, rates of change, standard deviations, and other statistics that might be meaningful to a trainer or veterinarian.

In one embodiment, a single parameter may be selected for display of information. The information displayed for the animal may include temperature, position, strain, and other sensed physiological parameters that together indicate an overall health or functioning of the animal at various times, such as during portions of a training run, at rest, racing conditions, etc. As previously indicated, patterns or collected data during such times may be processed and compared with known healthy or unhealthy patterns to perform diagnostics regarding current health, performance, or future problems that may occur based on the patterns.

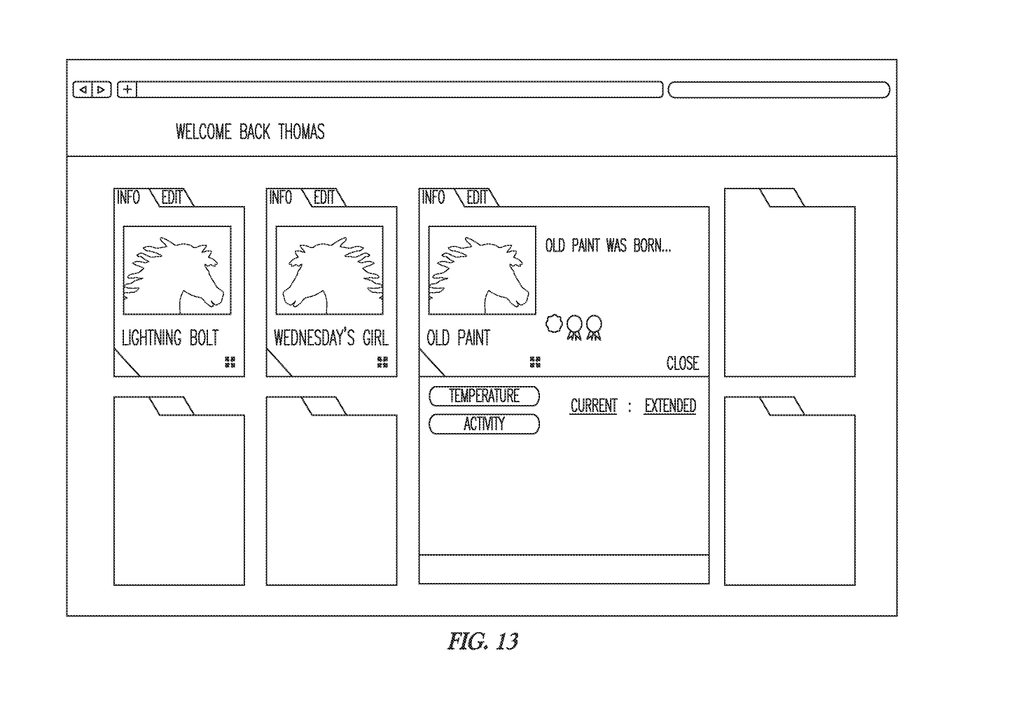

FIG. 13 is a web page based interface for selecting animals being monitored and providing additional information according to an example embodiment. The page illustrates further information on Old Paint, including a narrative describing the horse. The narrative could include past observations regarding training and data trends as well as any other desired information.

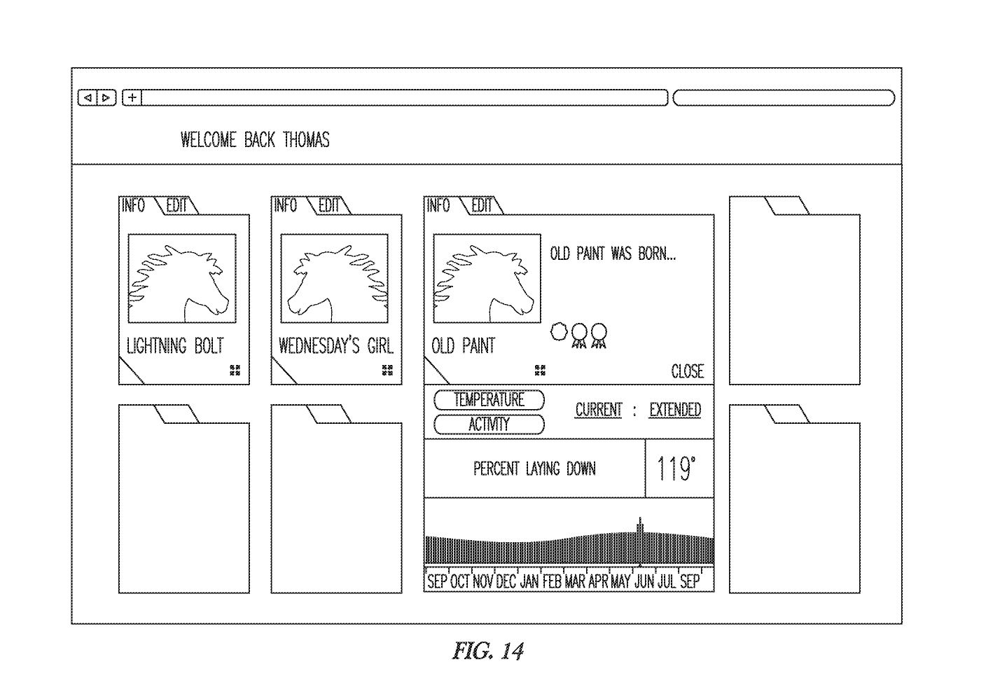

FIG. 14 is a web page based interface for selecting animals being monitored and providing postural information according to an example embodiment. This diagram includes a selected temperature sensor node with the current temperature listed and a history of percent of laying down over a one year plus period. The history is in the form of a histogram. The type of graph may be varied and different statistics may be shown in the graph in further embodiments.

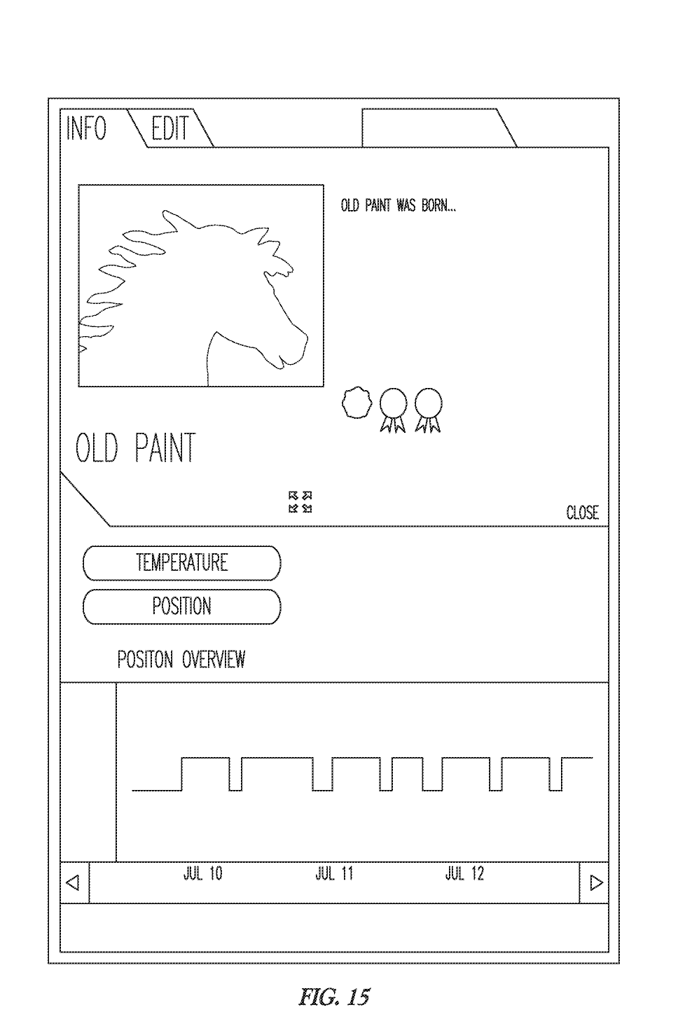

FIG. 15 is a web page based interface providing postural information in graphical form. A trend chart of temperature and postural behavior is provided.

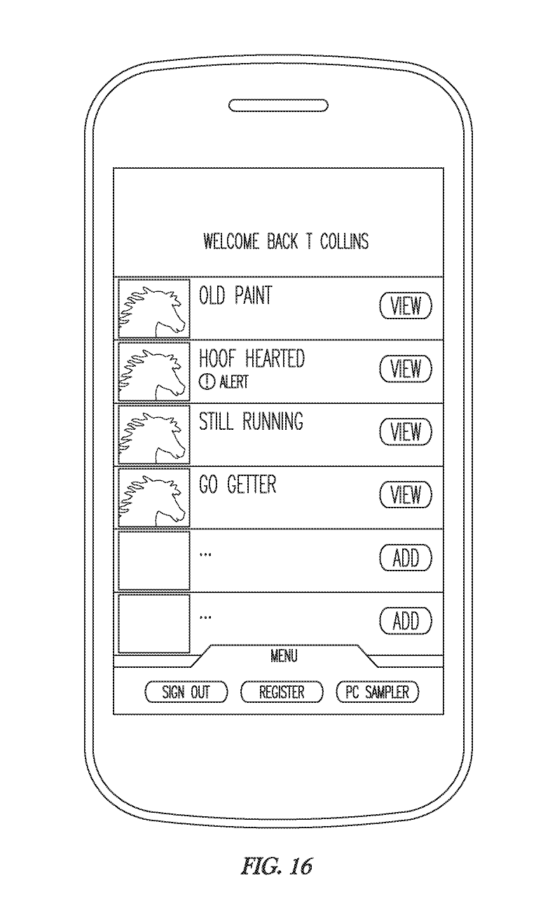

FIG. 16 is a mobile web page based interface for selecting animals being monitored according to an example embodiment. The interface is similar to that of FIG. 12 without a horse being currently selected. The user has the ability to select a displayed horse. The name of each horse and a picture may be provided on the selection menu.

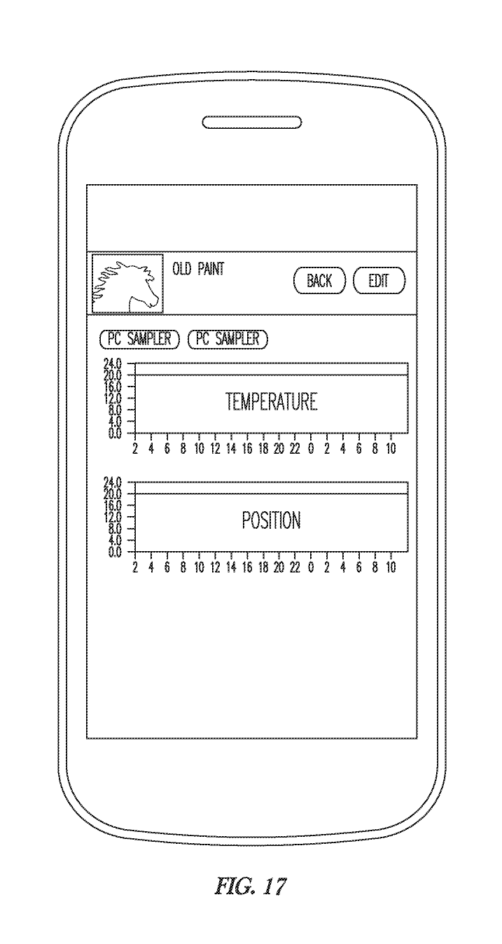

FIG. 17 is a mobile web page based interface illustrating temperature and position information for a selected animal according to an example embodiment. In this interface example, temporal postural data for the horse is provided.

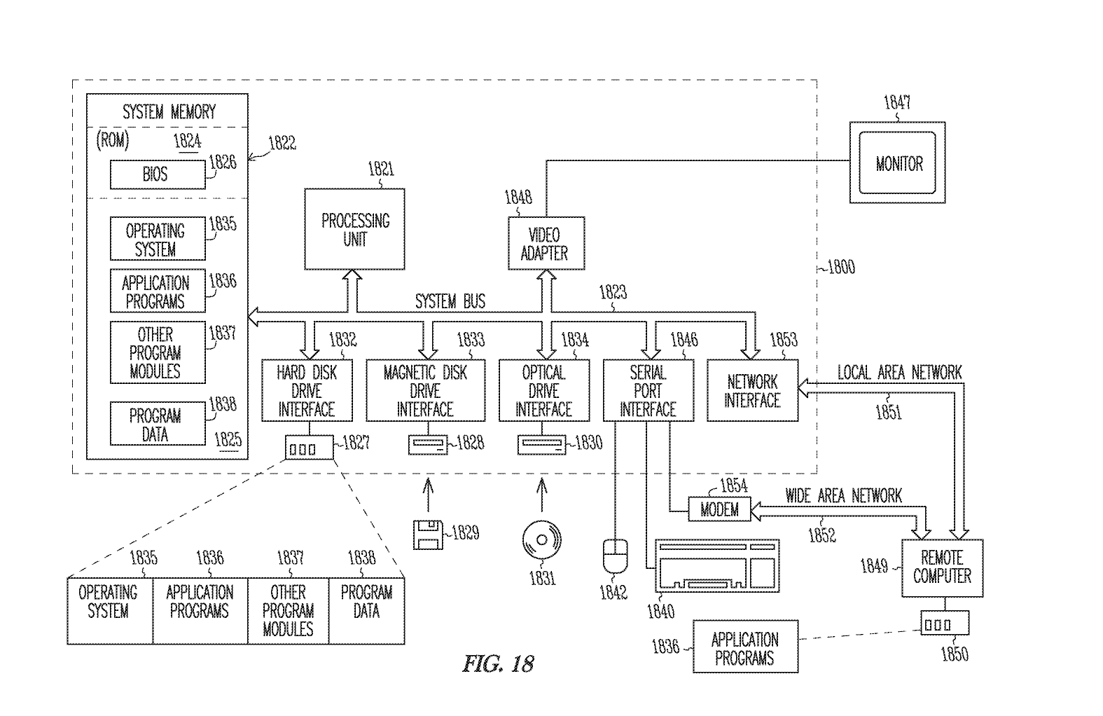

FIG. 18 is an example computer system for implementing one or more methods or algorithms according to an example embodiment. A hardware and operating environment is provided that may be applicable to execute drivers, compile and transmit sensor information to remote devices for viewing, receive sensor information directly from one or more sensors on a tail and transmit to a server, and other functions described herein. Many of the elements of FIG. 18 may be removed or reduced appropriate to the functions to be performed whether used in an integrated manner with the sensors to collect transmit information via an integrated transceiver, or to receive, process and retransmit information received from the transceiver.

As shown in FIG. 18, one embodiment of the hardware and operating environment includes a general purpose computing device in the form of a computer 1800 (e.g., a personal computer, workstation, or server), including one or more processing units 1821, a system memory 1822, and a system bus 1823 that operatively couples various system components including the system memory 1822 to the processing unit 1821. There may be only one or there may be more than one processing unit 1821, such that the processor of computer 1800 comprises a single central-processing unit (CPU), or a plurality of processing units, commonly referred to as a multiprocessor or parallel-processor environment. In various embodiments, computer 1800 is a conventional computer, a distributed computer, or any other type of computer.

The system bus 1823 can be any of several types of bus structures including a memory bus or memory controller, a peripheral bus, and a local bus using any of a variety of bus architectures. The system memory can also be referred to as simply the memory, and, in some embodiments, includes read-only memory (ROM) 1824 and random-access memory (RAM) 1825. A basic input/output system (BIOS) program 1826, containing the basic routines that help to transfer information between elements within the computer 1800, such as during start-up, may be stored in ROM 1824. The computer 1800 further includes a hard disk drive 1827 for reading from and writing to a hard disk, not shown, a magnetic disk drive 1828 for reading from or writing to a removable magnetic disk 1829, and an optical disk drive 1830 for reading from or writing to a removable optical disk 1831 such as a CD ROM or other optical media.

The hard disk drive 1827, magnetic disk drive 1828, and optical disk drive 1830 couple with a hard disk drive interface 1832, a magnetic disk drive interface 1833, and an optical disk drive interface 1834, respectively. The drives and their associated computer-readable media provide non-volatile storage of computer-readable instructions, data structures, program modules and other data for the computer 1800. It should be appreciated by those skilled in the art that any type of computer-readable media which can store data that is accessible by a computer, such as magnetic cassettes, flash memory cards, digital video disks, Bernoulli cartridges, random access memories (RAMs), read only memories (ROMs), redundant arrays of independent disks (e.g., RAID storage devices) and the like, can be used in the exemplary operating environment.

A plurality of program modules can be stored on the hard disk, magnetic disk 1829, optical disk 1831, ROM 1824, or RAM 1825, including an operating system 1835, one or more application programs 1836, other program modules 1837, and program data 1838. Programming for implementing one or more processes or method described herein may be resident on any one or number of these computer-readable media.

A user may enter commands and information into computer 1800 through input devices such as a keyboard 1840 and pointing device 1842. Other input devices (not shown) can include a microphone, joystick, game pad, satellite dish, scanner, or the like. These other input devices are often connected to the processing unit 1821 through a serial port interface 1846 that is coupled to the system bus 1823, but can be connected by other interfaces, such as a parallel port, game port, or a universal serial bus (USB). A monitor 1847 or other type of display device can also be connected to the system bus 1823 via an interface, such as a video adapter 1848. The monitor 1847 can display a graphical user interface for the user. In addition to the monitor 1847, computers typically include other peripheral output devices (not shown), such as speakers and printers.

The computer 1800 may operate in a networked environment using logical connections to one or more remote computers or servers, such as remote computer 1849. These logical connections are achieved by a communication device

coupled to or a part of the computer 1800; the invention is not limited to a particular type of communications device. The remote computer 1849 can be another computer, a server, a router, a network PC, a client, a peer device or other common network node, and typically includes many or all of the elements described above I/O relative to the computer 1800, although only a memory storage device 1850 has been illustrated. The logical connections depicted in FIG. 18 include a local area network (LAN) 1851 and/or a wide area network (WAN) 1852. Such networking environments are commonplace in office networks, enterprise-wide computer networks, intranets and the internet, which are all types of networks.

When used in a LAN-networking environment, the computer 1800 is connected to the LAN 1851 through a network interface or adapter 1853, which is one type of communications device. In some embodiments, when used in a WAN-networking environment, the computer 1800 typically includes a modem 1854 (another type of communications device) or any other type of communications device, e.g., a wireless transceiver, for establishing communications over the wide-area network 1852, such as the internet. The modem 1854, which may be internal or external, is connected to the system bus 1823 via the serial port interface 1846. In a networked environment, program modules depicted relative to the computer 1800 can be stored in the remote memory storage device 1850 of remote computer, or server 1849. It is appreciated that the network connections shown are exemplary and other means of, and communications devices for, establishing a communications link between the computers may be used including hybrid fiber-coax connections, T1-T3 lines, DSL's, OC-3 and/or OC-12, TCP/IP, microwave, wireless application protocol, and any other electronic media through any suitable switches, routers, outlets and power lines, as the same are known and understood by one of ordinary skill in the art.

The following statements are provided as examples, of various embodiments.

1. A system comprising:

a physiological property sensor; and

circuitry to collect information from the physiological property sensor and transmit the information to a server, wherein the physiological property sensor and circuitry are formed as an integrated patch adapted to be placed to sense a physiological property on a skin area on a tail of an animal.

2. The system of example 1 wherein the physiological property sensor is shaped to be positioned on the skin area on an underside of the tail of a horse.

3. The system of example 2 wherein the integrated node is shaped to be placed on a flexible adhesive patch such that it is operable to sense a pulse.

4. The system of any of examples 2-3 wherein the integrated node is shaped to be placed on a flexible adhesive patch such that it is operable to sense altitude.

5. The system of any of examples 2-4 wherein the integrated node is shaped to be placed on a flexible adhesive patch such that it is operable to sense posture.

6. The system of any of examples 1-5 wherein the circuitry comprises a microcontroller and a transmitter.

7. A system comprising:

a physiological property sensor; and

circuitry to collect information from the physiological property sensor and transmit the information to a server, wherein the physiological property sensor and circuitry are formed as an integrated patch adapted to be placed to sense a physiological property on a hoof wall of an animal.

8. A system comprising:

a server;

a temperature sensor to sense ambient temperature about an animal and provide data representative of the sensed ambient temperature to the server;

a physiological property sensor; and

circuitry to collect information from the physiological property sensor and transmit the information to the server, wherein the physiological property sensor and circuitry are formed as an integrated node adapted to be placed on a tail of the animal to sense temperature of the animal, and wherein the server is a particular machine programmed to compile received information, adjust the sensed animal temperature as a function of sensed ambient temperature, and send communications to a device with a display.

9. The system of example 8 wherein compiling the received information includes determining trends in measured physiological properties.

10. The system of example 9 wherein compiling the received information includes matching measured physiological properties to predefined patterns of physiological properties.

11. The system of any of examples 8-10 wherein the communications include real time temperature information that is viewable while watching the animal.

12. The system of any of examples 8-11 wherein the communications include real time temperature information that is analyzed by the server and wherein the communications include an alert of an aberration sent to the device.

13. The system of any of examples 8-12 and further a posture sensor to detect posture of the animal and transmit information about the posture to the server.

14. A method comprising:
receiving at a server, sensed temperature information via a sensor coupled to a skin area on a tail of an animal;
receiving ambient temperature information corresponding to an ambient temperature about the animal; and

processing the received sensed temperature information by the server to adjust the sensed temperature as a function of an ambient temperature about the animal.

15. The method of example 14 and further comprising transmitting temperature information to a remote device for real time viewing of the temperature information.

16. The method of any of examples 14-15 and further comprising receiving sensed posture information to identify whether the animal is standing or lying down.

17. The method of example 14 wherein the temperature information corresponds to a rectal temperature of a horse.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

The following statements are potential claims that may be converted to claims in a future application. No modification of the following statements should be allowed to affect the interpretation of claims which may be drafted when this provisional application is converted into a regular utility application.

1. A system comprising:
a physiological property sensor; and
circuitry to collect information from the physiological property sensor and transmit the information to a server, wherein the physiological property sensor and circuitry are formed as an integrated patch adapted to be placed to sense a physiological property on a skin area on a tail of an animal.

2. The system of claim 1 wherein the physiological property sensor is shaped to be positioned on the skin area on an underside of the tail of a horse.

3. The system of claim 2 wherein the integrated node is shaped to be placed on a flexible adhesive patch such that it is operable to sense a pulse.

4. The system of claim 2 wherein the integrated node is shaped to be placed on a flexible adhesive patch such that it is operable to altitude.

5. The system of claim 2 wherein the integrated node is shaped to be placed on a flexible adhesive patch such that it is operable to sense posture.

6. The system of claim 1 wherein the circuitry comprises a microcontroller and a transmitter.

7. A system comprising:
a physiological property sensor; and
circuitry to collect information from the physiological property sensor and transmit the information to a server, wherein the physiological property sensor and circuitry are formed as an integrated patch adapted to be placed to sense a physiological property on a hoof wall of an animal.

8. A system comprising:
a server;
a temperature sensor to sense ambient temperature about an animal and provide data representative of the sensed ambient temperature to the server;
a physiological property sensor; and
circuitry to collect information from the physiological property sensor and transmit the information to the server, wherein the physiological property sensor and circuitry are formed as an integrated node adapted to be placed on a tail of the animal to sense temperature of the animal, and wherein the server is a particular machine programmed to compile received information, adjust the sensed animal temperature as a function of sensed ambient temperature, and send communications to a device with a display.

9. The system of claim 8 wherein compiling the received information includes determining trends in measured physiological properties.

10. The system of claim 9 wherein compiling the received information includes matching measured physiological properties to predefined patterns of physiological properties.

11. The system of claim 8 wherein the communications include real time temperature information that is viewable while watching the animal.

12. The system of claim 8 wherein the communications include real time temperature information that is analyzed by the server and wherein the communications include an alert of an aberration sent to the device.

13. The system of claim 8 and further comprising a posture sensor to detect posture of the animal and transmit information about the posture to the server.

14. A method comprising:
receiving at a server, sensed temperature information via a sensor coupled to a skin area on a tail of an animal;
receiving ambient temperature information corresponding to an ambient temperature about the animal; and
processing the received sensed temperature information by the server to adjust the sensed temperature as a function of an ambient temperature about the animal.

15. The method of claim 14 and further comprising transmitting temperature information to a remote device 1 for real time viewing of the temperature information.

16. The method of claim 14 and further comprising receiving sensed posture information to identify whether the animal is standing or lying down.

17. The method of claim 14 wherein the temperature information corresponds to a rectal temperature of a horse.
